SEQUENCE LISTING (17 sequences)

SEQ ID NO: 1 - DNA, 40 bp, Artificial Sequence
  Description of Artificial SequencePCR forward primer OATPF-1F
actgtaaaacgacggccagtaatgaggcttaaactgggca

SEQ ID NO: 2 - DNA, 40 bp, Artificial Sequence
  Description of Artificial SequencePCR reverse primer OATPF-1R
accaggaaacagctatgaccggtagagattgcttgcaccg

SEQ ID NO: 3 - DNA, 20 bp, Artificial Sequence
  Description of Artificial SequenceValidation primer
tggacacttcatccaaagaa

SEQ ID NO: 4 - DNA, 41 bp, Artificial Sequence
  Description of Artificial SequencePCR forward primer OATPF-2F
actgtaaaacgacggccagtctatgagccagatcttctggc

SEQ ID NO: 5 - DNA, 43 bp, Artificial Sequence
  Description of Artificial SequencePCR reverse primer OATPF-2R
accaggaaacagctatgacccagaagctttgaaagattttccc

SEQ ID NO: 6 - DNA, 20 bp, Artificial Sequence
  Description of Artificial SequenceValidation primer
tattctccttcctccaattc

SEQ ID NO: 7 - DNA, 40 bp, Artificial Sequence
  Description of Artificial SequencePCR forward primer OATPF-3F
actgtaaaacgacggccagttgggcacctaattgctacct

SEQ ID NO: 8 - DNA, 40 bp, Artificial Sequence
  Description of Artificial SequencePCR reverse primer OATPF-3R
accaggaaacagctatgacctgagggaacatacccttggt

SEQ ID NO: 9 - DNA, 20 bp, Artificial Sequence
  Description of Artificial SequenceValidation primer
atcagtgtgtgtggagctgc

SEQ ID NO: 10 - DNA, 40 bp, Artificial Sequence
  Description of Artificial SequencePCR forward primer OATPF-4F
actgtaaaacgacggccagtgctgttctaggcaaacaggg

SEQ ID NO: 11 - DNA, 16 bp, Artificial Sequence
  Description of Artificial SequencePCR reverse primer OATPF-4R
ccagtagttgggttgt

SEQ ID NO: 12 - DNA, 20 bp, Artificial Sequence
  Description of Artificial SequenceValidation primer
cttcttctagacatatatat

SEQ ID NO: 13 - DNA, 40 bp, Artificial Sequence
  Description of Artificial SequencePCR forward primer OATPF-5F
actgtaaaacgacggccagtcttcagctctttctgtgccc

SEQ ID NO: 14 - DNA, 41 bp, Artificial Sequence
  Description of Artificial SequencePCR reverse primer OATPF-5R
accaggaaacagctatgaccttccatcaaactaatgagggg

SEQ ID NO: 15 - DNA, 54 bp, Homo sapiens
atttttactttaaaaactaactttgacagatcagagtcaaggaatgtgtttata

SEQ ID NO: 16 - DNA, 3077 bp, Homo sapiens
cattgaaaggaaatggctatctttgatctcttcctccagatcagagtcaaggaatgtgtttataatggacacttcatccaaagaaaatatccagttgttctgcaaaacttcagtgcaacctgttggaaggccttcttttaaaacagaatatccctcctcagaagaaaagcaaccatgctgtggtgaactaaaggtgttcttgtgtgccttgtcttttgtttactttgccaaagcattggcagaaggctatctgaagagcaccatcactcagatagagagaaggtttgatatcccttcttcactggtgggagttattgatggtagttttgaaattgggaatctcttagttataacatttgttagctactttggagccaaacttcacaggccaaaaataattggagcagggtgtgtaatcatgggagttggaacactgctcattgcaatgcctcagttcttcatggagcagtacaaatatgagagatattctccttcctccaattccactctcagcatctctccgtgtctcctagagtcaagcagtcaattaccagtttcagttatggaaaaatcaaaatccaaaataagtaacgaatgtgaagtggacactagctcttccatgtggatttatgttttcctgggcaatcttcttcgtggaataggagaaactcccattcagcctttgggcattgcctacctggatgattttgccagtgaagacaatgcagctttctatattgggtgtgtgcagacggttgcaattataggaccaatctttggtttcctgttaggctcattatgtgccaaactatatgttgacattggctttgtaaacctagatcacataaccattaccccaaaagatccccagtgggtaggagcctggtggcttggctatctaatagcaggaatcataagtcttcttgcagctgtgcctttctggtatttaccaaagagtttaccaagatcccaaagtagagaggattctaattcttcctctgagaaatccaagtttattatagatgatcacacagactaccaaacaccccagggagaaaatgcaaaaataatggaaatggcaagagattttcttccatcactgaagaatctttttggaaacccagtatacttcctatatttatgtacaagcactgttcagttcaattctctgttcggcatggtgacgtacaaaccaaagtacattgagcagcagtatggacagtcatcctccagggccaactttgtgatcgggctcatcaacattccagcagtggcccttggaatattctctggggggatagttatgaaaaaattcagaatcagtgtgtgtggagctgcaaaactctacttgggatcatctgtctttggttacctcctatttctttccctgtttgcactgggctgtgaaaattctgatgtggcaggactaactgtctcctaccaaggaaccaaacctgtctcttatcatgaacgagctctcttttcagattgcaactcaagatgcaaatgttcagagacaaaatgggaacccatgtgcggtgaaaatggaatcacatatgtatcagcttgtcttgctggttgtcaaacctccaacaggagtggaaaaaatattatattttacaactgcacttgtgtgggaattgcagcttctaaatccggaaattcctcaggcatagtgggaagatgtcagaaagacaatggatgtccccaaatgtttctgtatttccttgtaatttcagtcatcacatcctatactttatccctaggtggcatacctggatacatattacttctgaggtgcattaagccacagcttaagtcttttgccttgggtatctacacattagcaataagagttcttgcaggaatcccagctccagtgtattttggagttttgattgatacttcatgcctcaaatggggatttaaaagatgtggaagtagaggatcatgcagattatatgattcaaatgtcttcagacatatatatctgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaacatataatggaagatgcagatgataaaactaattttgaactttttaatttatataaattattttatatcacttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtgtattttaataatgatcttaggatggagcagaacatggagaggaagatttcattttaagctcctccttttctttgaaatacaataatttatatagaaatgtgtagcagcaaattatattggggattagaattttgaattaatagctctcctactattaatttacatgtgctttttgtgtggcgctataagtgactatggttgtaaagtaataaaattgatgttaacatgcccaattattgttcttttatgaattcaatgaatttaaaactattgttaaatataatactgccccactttaatatatgtaagcaacttcctacttatacacgacgtgttcctaaaacatgtttgaaaggtgaatttctgaaagtctacaataaatgtaggtgttacaacaggaaaaaaaaaaaaaaaaaa

SEQ ID NO: 17 - protein, 712 aa, Homo sapiens
MDTSSKENIQLFCKTSVQPVGRPSFKTEYPSSEEKQPCCGELKVFLCALSFVYFAKALAEGYLKSTITQIERRFDIPSSLVGVIDGSFEIGNLLVITFVSYFGAKLHRPKIIGAGCVIMGVGTLLIAMPQFFMEQYKYERYSPSSNSTLSISPCLLESSSQLPVSVMEKSKSKISNECEVDTSSSMWIYVFLGNLLRGIGETPIQPLGIAYLDDFASEDNAAFYIGCVQTVAIIGPIFGFLLGSLCAKLYVDIGFVNLDHITITPKDPQWVGAWWLGYLIAGIISLLAAVPFWYLPKSLPRSQSREDSNSSSEKSKFIIDDHTDYQTPQGENAKIMEMARDFLPSLKNLFGNPVYFLYLCTSTVQFNSLFGMVTYKPKYIEQQYGQSSSRANFVIGLINIPAVALGIFSGGIVMKKFRISVCGAAKLYLGSSVFGYLLFLSLFALGCENSDVAGLTVSYQGTKPVSYHERALFSDCNSRCKCSETKWEPMCGENGITYVSACLAGCQTSNRSGKNIIFYNCTCVGIAASKSGNSSGIVGRCQKDNGCPQMFLYFLVISVITSYTLSLGGIPGYILLLRCIKPQLKSFALGIYTLAIRVLAGIPAPVYFGVLIDTSCLKWGFKRCGSRGSCRLYDSNVFRHIYLGLTVILGTVSILLSIAVLFILKKNYVSKHRSFITKRERTMVSTRFQKENYTTSDHLLQPNYWPGKETQL

(19) United States
(12) Patent Application Publication
(10) Pub. No.: US 2007/0122803 A1
(43) Pub. Date: May 31, 2007

(54) Methods for the detection of polymorphisms in the human oatpf gene

(76) Inventors: Rachael Moore, Cheshire (GB); Adam Jeston Dudley, Willington, DE (US)

Correspondence Address: FISH & RICHARDSON P.C., P.O BOX 1022, MINNEAPOLIS, MN, 55440-1022, US

(21) Appl. No.: 10/517,647

(22) PCT Filed: Jun. 10, 2003
(86) PCT No.: PCT/GB03/02487

§ 371 Date: Dec. 10, 2004

Related U.S. Application Data
(60) Provisional application No. 60/388,692, filed on Jun. 14, 2002.

(30) Foreign Application Priority Data
Jun. 13, 2002 (GB) 0213580.4

(51) Int. Cl. C12Q 1/68; G01N 33/53; C07H 21/04; C07K 16/40

(52) U.S. Cl. 435/6; 536/24.3; 435/7.1; 530/388.26

(57) ABSTRACT

The invention provides a method for the detection of a polymorphism in OATPF in a human. The method for detection of a nucleic acid polymorphism is selected from amplification refractory mutation system and restriction fragment length polymorphism. The invention also provides use of the method to assess the pharmacogenetics of a drug transportable by OATF.

This invention relates to polymorphisms in the human OATPF gene and corresponding novel allelic polypeptides encoded thereby. The invention also relates to methods and materials for analysing allelic variation in the OATPF gene, and to the use of OATPF polymorphism in treatment of diseases with OATPF transportable drugs.

Membrane transporters are important for the absorption of oral medications across the gastrointestinal tract, uptake in to target tissues such as the liver or brain, and excretion into the bile and urine. Changes in the activities of transporters may therefore have a significant effect on the bioavailability of clinically important drugs.

It has been reported in the literature that polymorphisms in proteins involved in drug transport can alter the function of the protein. For example, the multidrug-resistance (MDR-1) gene contains a polymorphism in exon 26 (C3435T) which has been correlated with expression levels and function of MDR-1. Individuals homozygous for this polymorphisms have significantly lower duodenal MDR-1 expression and high digoxin plasma levels, suggesting this polymorphism affects the absorption and tissue concentrations of substrates of MDR-1 (S Hoffmeyer et al. Proceedings National Academy Science (2000) 97, 3473-3478).

The human sodium independent organic anion transporting polypeptide (OATP) F gene is a member of the OATP supergene family involved in multifunctional transport of organic anions (I. Tamai et al. Biochemical and Biophysical Research Communications 273, 251-260 (2000); M. Kusuhara &Y. Sugiyama Journal of Controlled Release 78 (2002) 43-54). There is an alternative nomenclature for this family as SLC21A (solute carriers) and OATPF relates to SLC21A14. A cDNA sequence encoding OATPF has been submitted to the EMBL database under accession number AF260704. A cDNA sequence encoding for OATPF has also been submitted to the EMBL database under accession number AF205076, and has been accorded the alternative name for OATPF of OATPRP5.

OATPF has a 43% identity at the amino acid level with its gene family member human OATPC (SLC21A6). OATPC has been shown to be involved in the transport of drugs involved in lipid lowering e.g. statins (D. Nakai et al. J Pharmacol Exp Ther 2001 297: 861-867). Statins have been referred to as a first-line therapy for patients with atherosclerotic vascular diseases (B. Hsiang et al. J. Biol Chem 274, 37161-37168 (1999)). Due to its sequence homology, it is likely that OATPF may transport similar substrates as OATPC. OATPF is also homologous to a rat gene named BSAT1. BSAT1 is expressed at the blood-brain barrier in rats and human homologues of this gene may be important in transport of pharmaceutical agents into the brain.

DNA polymorphisms are variations in DNA sequence between one individual and another. DNA polymorphisms may lead to variations in amino acid sequence and consequently to altered protein structure and functional activity. Polymorphisms may also affect mRNA synthesis, maturation, transportation and stability. Polymorphisms which do not result in amino acid changes (silent polymorphisms) or which do not alter any known consensus sequences may nevertheless have a biological effect, for example by altering mRNA folding or stability.

Knowledge of polymorphisms may be used to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The first 54bp of genomic DNA sequence immediately upstream of the OATPF protein coding sequence is set out as SEQ ID NO: 15, with the first nucleotide of the genomic DNA sequence accorded position 1. The position of the polymorphism in the genomic DNA sequence is defined with reference to SEQ ID NO: 15 unless stated otherwise or apparent from the context.

A cDNA sequence encoding OATPF is set out as SEQ ID NO: 16, with the first nucleotide of the OATPF coding region accorded position 65. All positions of polymorphisms in the human OATPF gene transcribed into messenger RNA (and thence cDNA) herein refer to the positions in SEQ ID NO: 16 unless stated otherwise or apparent from the context.

All positions herein of polymorphisms in the OATPF polypeptide are defined with reference to SEQ ID NO: 17 unless stated otherwise or apparent from the context.

The present invention is based on the discovery of four polymorphisms in the human OATPF gene and one polymorphism in the genomic DNA sequence immediately upstream of the human OATPF gene. The polymorphisms of the present invention may have a functional effect on the protein and hence alter the transport of pharmaceutical agents.

According to one aspect of the present invention there is provided a method for the detection of a polymorphism in OATPF in a human, which method comprises determining the sequence of the human at any one of the following positions: position 11-16 of SEQ ID NO: 15; positions 86, 505, 1339, 1991 of SEQ ID NO: 16; position 8 of SEQ ID NO: 17.

The term “human” includes both a human having or suspected of having an OATPF mediated response to a drug and an asymptomatic human who may be tested for predisposition or susceptibility to such a response. At each position the human may be homozygous for an allele or the human may be a heterozygote.

The term “detection of a polymorphism” refers to determination of the genetic status of an individual at a polymorphic position (in which the individual may be homozygous or heterozygous at each position).

The term “OATPF mediated response” means any disease in which changing the level of an OATPF mediated response or changing the biological activity of OATPF would be of therapeutic benefit.

The term “polymorphism” includes nucleotide substitution, nucleotide insertion and nucleotide deletion, which in the case of insertion and deletion includes insertion or deletion of one or more nucleotides at a position of a gene and variable numbers of a repeated DNA sequence.

In one embodiment of the invention preferably the polymorphism is further defined as: polymorphism at position 11-16 is presence of TAAAAA and/or insertion of
ACTTTGAAAG in lieu thereof;
polymorphism at position 86 is presence of A and/or G;
polymorphism at position 505 is presence of C and/or T;
polymorphism at position 1339 is presence of A and/or G;
polymorphism at position 1991 is presence of A and/or T; and
polymorphism at position 8 is presence of Asn and/or Asp.

The polymorphism at position 11-16 of SEQ ID NO: 15 is the result of a deletion-insertion event defined as deletion of bases 11-16 of SEQ ID NO: 15 and insertion of ACTTTGAAAG in lieu thereof. This results in an overall extra four bases and it will be appreciated by the skilled person that this will have an effect on the numbering of positions downstream of this. For example, position 17 of SEQ ID NO: 15 becomes position 21 after the deletion-insertion event. It will also be appreciated by the skilled person that it may not be necessary to sequence the entire 11-16 bases or the insertion at this position to distinguish between the two alleles. For example, position 11 is either a T or an A when comparing the sequence of the two alleles.

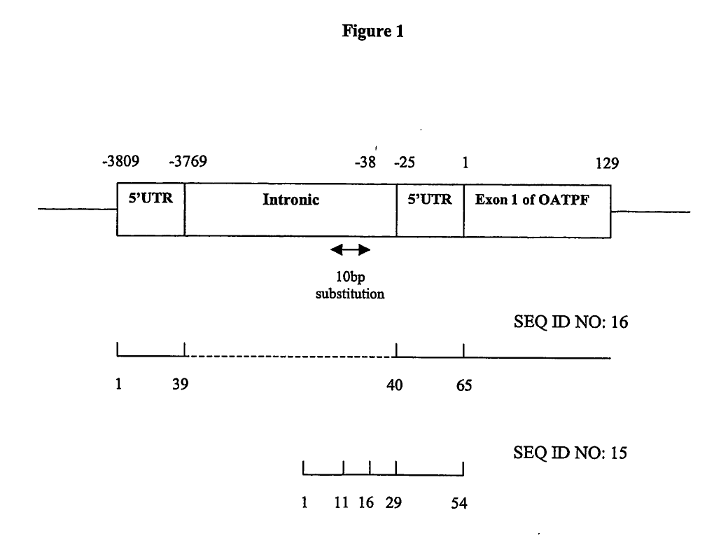

In FIG. 1, the polymorphism at position 11-16 is shown relative to the +1 ATG start site of the OATPF gene (position 65 as defined in SEQ ID NO: 16). Splicing of the genomic DNA sequence of FIG. 1 results in excision of the intronic region to produce a spliced 5′ UTR (position 1-64 as defined in SEQ ID NO: 16) immediately upstream of the OATPF gene.

Preferred methods for detection of nucleic acid polymorphism are amplification refractory mutation system and restriction fragment length polymorphism.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, ₂nd Edition by Newton & Graham, BIOS Scientific Publishers Limited, 1997.

Abbreviations:

TABLE 1
Mutation Detection Techniques

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| DHPLC | Denaturing high performance liquid chromotography |
| FRET | Fluorescence resonance energy transfer |
| LCR | Ligase chain reaction |
| MASDA | Multiple allele specific diagnostic assay |
| NASBA | Nucleic acid sequence based amplification |
| OATP | Na+-independent organic anion transporting polypeptide |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |
| UTR | Untranslated region |

General: DNA sequencing, Sequencing by hybridisation
Scanning: PTT*, SSCP, DGGE, TGGE, Cleavase, Heteroduplex analysis, CMC, Enzymatic mismatch cleavage
*Note:
not useful for detection of promoter polymorphisms.

Hybridisation Based
Solid phase hybridisation: Dot blots, MASDA, Reverse dot blots, Oligonucleotide arrays (DNA Chips)
Solution phase hybridisation: Taqman™ —U.S. Pat. Nos. 5,210,015 & 5,487,972 (Hoffmann-La Roche), Molecular Beacons—Tyagi et al (1996), Nature Biotechnology, 14, 303; WO 95/13399 (Public Health Inst., New York)
Extension Based: ARMS™, ALEX™ —European Patent No. EP 332435 B1 (Zeneca Limited), COPS—Gibbs et al (1989), Nucleic Acids Research, 17, 2347.
Incorporation Based: Mini-sequencing, APEX
Restriction Enzyme Based: RFLP, Restriction site generating PCR
Ligation Based: OLA
Other: Invader assay
   Table 2—Signal Generation or Detection Systems
Fluorescence: FRET, Fluorescence quenching, Fluorescence polarisation—United Kingdom Patent No. 2228998 (Zeneca Limited)
Other: Chemiluminescence, Electrochemiluminescence, Raman, Radioactivity, Colorimetric, Hybridisation protection assay, Mass spectrometry
   Table 3—Further Amplification Methods
SSR, NASBA, LCR, SDA, b-DNA
   Table 4—Protein Variation Detection Methods
Immunoassay
Immunohistology
Peptide sequencing

Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom. Histological techniques are known in the art e.g. Theory and Practice of Histological Techniques, 4^{th} Edition, edited by J D Bancroft and A Stevens, Churchill Livingstone, 1996. Protein sequencing is described in Laboratory techniques Biochemistry and Molecular Biology, Volume 9, Sequencing of Proteins and Polypeptides, G Allen, 2^{nd} revised edition, Elsevier, 1989.

Preferred mutation detection techniques include ARMS™, ALEX™, COPS, Taqman, Molecular Beacons, RFLP, restriction site based PCR and FRET techniques, polyacrylamide gel electrophoresis and capillary electrophoresis.

Particularly preferred methods include ARMS™ and RFLP based methods. ARMS™ is an especially preferred method.

In a further aspect, the methods of the invention are used to assess the pharmacogenetics of a drug transportable by OATPF.

Assays, for example reporter-based assays, may be devised to detect whether one or more of the above polymorphisms affect transcription levels and/or message stability.

Individuals who carry particular allelic variants of the OATPF gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The methods of the invention may be useful both to predict the clinical response to such agents and to determine therapeutic dose.

In a further aspect, the methods of the invention, are used to assess the predisposition and/or susceptibility of an individual to diseases mediated by OATPF. The present invention may be used to recognise individuals who are particularly at risk from developing such diseases.

In a further aspect, the methods of the invention are used in the development of new drug therapies which selectively target one or more allelic variants of the OATPF gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further aspect of the invention the presence or absence of variant nucleotides is detected by reference to the loss or gain of, optionally engineered, sites recognised by restriction enzymes.

According to another aspect of the present invention there is provided a human OATPF gene or its complementary strand comprising a variant allelic polymorphism at one or ore of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 contiguous bases of the human OATPF gene and comprising an allelic variant selected from any of the following:

| Region | variant | Position |
|---|---|---|
| Exon 1 | G | 86 (SEQ ID NO: 16) |
| Exon 4 | T | 505 (SEQ ID NO: 16) |
| Exon 9 | G | 1339 (SEQ ID NO: 16) |
| Exon 14 | T | 1991 (SEQ ID NO: 16) |
| Intronic | ACTTTGAAAG | 11-16 (SEQ ID NO: 15) |

According to another aspect of the present invention there is provided a human OATPF gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting an OATPF gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting an OATPF gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting an OATPF gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the polymorphisms of this invention may be used as genetic markers in linkage studies. This particularly applies to the polymorphisms of relatively high frequency. The OATPF gene is on chromosome 12. Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that polymorphisms with low frequency alleles are particularly useful as markers of low frequency haplotypes. As particular mutations or polymorphisms associated with certain clinical features, such as adverse or abnormal events, are likely to be of low frequency within the population, low frequency polymorphisms may be particularly useful in identifying these mutations (for examples see: De Stefano V et al. Ann Hum Genet (1998) 62:481-90; and Keightley A M et al. Blood (1999) 93:4277-83).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis or any other bioinformatic analysis. The reader is referred to Bioinformatics, A practical guide to the analysis of genes and proteins, Edited by A D Baxevanis & B F F Ouellette, John Wiley & Sons, 1998. Any computer readable medium may be used, for example, compact disk, tape, floppy disk, hard drive or computer chips.

The polynucleotide sequences of the invention, or parts thereof, particularly those relating to and identifying the polymorphisms identified herein represent a valuable information source, for example, to characterise individuals in terms of haplotype and other sub-groupings, such as investigation of susceptibility to treatment with particular drugs. These approaches are most easily facilitated by storing the sequence information in a computer readable medium and then using the information in standard bioinformatics programs or to search sequence databases using state of the art searching tools such as “GCC”. Thus, the polynucleotide sequences of the invention are particularly useful as components in databases useful for sequence identity and other search analyses. As used herein, storage of the sequence information in a computer readable medium and use in sequence databases in relation to ‘polynucleotide or polynucleotide sequence of the invention’ covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data

The invention provides a computer readable medium having stored thereon one or more polynucleotide sequences of the invention. For example, a computer readable medium is provided comprising and having stored thereon a member selected from the group consisting of: a polynucleotide comprising the sequence of a polynucleotide of the invention, a polynucleotide consisting of a polynucleotide of the invention, a polynucleotide which comprises part of a polynucleotide of the invention, which part includes at least one of the polymorphisms of the invention, a set of polynucleotide sequences wherein the set includes at least one polynucleotide sequence of the invention, a data set comprising or consisting of a polynucleotide sequence of the invention or a part thereof comprising at least one of the polymorphisms identified herein.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e. screen for the presence of a polymorphism.

According to another aspect of the present invention there is provided a method of treating a human in need of treatment with a drug transportable by OATPF in which the method comprises:
i) detection of a polymorphism in OATPF in a human, which method comprises determining the sequence of the human at one or more of: position 15-24 of SEQ ID NO: 15; positions 86, 505, 1339 and 1991 of SEQ ID NO: 16; position 8 of SEQ ID NO: 17; and
ii) administering an effective amount of the drug.

Preferably determination of the status of the human is clinically useful. Examples of clinical usefulness include deciding which statin drug or drugs to administer and/or in deciding on the effective amount of the statin drug or drugs. Statins already approved for use in humans include atorvastatin, cerivastatin, fluvastatin, pravastatin and simvastatin. Statins under development include rosuvastatin. The reader is referred to the following references for further information: Drugs and Therapy Perspectives (12^{th} May 1997), 9: 1-6; Chong (1997) Pharmacotherapy 17: 1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368; Olsson AG, Pears J, McKellar J, Mizan J & Raza A (2001) American Journal of Cardiology 88(5): 504-508. The term “drug transportable by OATPF” means that transport by OATPF in humans is an important part of a drug exerting its pharmaceutical effect in man. For example, some statins have to be transported to the liver by OATPC, which is highly homologous to OATPF, to exert their lipid lowering effects. Accordingly, OATPF is expected to be involved in statin transport.

According to another aspect of the present invention there is provided use of a drug transportable by OATPF in preparation of a medicament for treating a disease in a human determined as having a polymorphism defined herein.

According to another aspect of the present invention there is provided a pharmaceutical pack comprising OATPF transportable drug and instructions for administration of the drug to humans tested for a polymorphism described therein, preferably at one or more of the positions defined herein.

One of the polymorphisms of the present invention result in variation in the amino acid sequence of the translated protein. Polymorphism at position 86 as defined in SEQ ID NO: 16 results in an amino acid change from asparagine to aspartic acid at corresponding position 8 of the translated protein (Asn8Asp) as defined in SEQ ID NO: 17.

Thus according to another aspect of the present invention there is provided an allelic variant of human OATPF polypeptide having an aspartic acid at position 8 of SEQ ID NO: 17 or a fragment thereof comprising at least 10 amino acids provided that the fragment comprises the allelic variant at position 8 of SEQ ID NO: 17.

Fragments of OATPF polypeptide are at least 10 amino acids, more preferably at least 15 amino acids, more preferably at least 20 amino acids. The polypeptides of the invention do not encompass naturally occurring polypeptides as they occur in nature, for example, the polypeptide is at least partially purified from at least one component with which it occurs naturally. Preferably the polypeptide is at least 30% pure, more preferably at least 60% pure, more preferably at least 90% pure, more preferably at least 95% pure, and more preferably at least 99% pure.

According to another aspect of the present invention there is provided an antibody specific for an allelic variant of human OATPF polypeptide having an aspartic acid at position 8 of SEQ ID NO: 17 or a fragment thereof comprising at least 10 amino acids provided that the fragment comprises the allelic variant at position 8 of SEQ ID NO: 17.

Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the allelic variant of OATPF with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following Examples and FIG. 1 is a schematic representation of the genomic DNA sequence upstream of Exon 1 of the OATPF gene, and shown relative to the corresponding nucleotide positions of SEQ ID NO: 15 and SEQ ID NO: 16. All temperatures are in degrees Celsius.

In the Examples below, unless otherwise stated, the following methodology and materials have been applied.

AMPLITAQ™, available from Perkin-Elmer Cetus, may be used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or in “Current Protocols in Molecular Biology Volumes1-3, edited by F M Asubel, R Brent and R E Kingston; published by John Wiley, 1998.

### EXAMPLES

### Example 1

Identification of Polymorphisms

1. Methods

The cDNA sequence of OATPF (AF260704) was used to identify the genomic position of the gene and obtain genomic DNA sequence. The genomic DNA sequence was used to design PCR primers to amplify over the exons and intron/exon boundaries of the full length of the OATP gene. Twenty-nine individual Caucasian genomic DNA samples were used as templates for PCR amplification. The products were then sequenced by dye-primer sequencing using Amersham's MegaBACE™ technology. The alignment of sequence traces enabled the identification of polymorphisms. The frequency of the polymorphisms was confirmed using genomic DNA from 20 individuals by primer extension analysis by DHPLC using the WAVE® system by Transgenomics and also by sequencing as described above. The deletion-insertion event was confirmed by sequencing in the forward and reverse direction.

PCR Products

| | | | Primer used for |
|---|---|---|---|
| | PCR forward | | confirmation of |
| Polymorphism | oligo | PCR reverse oligo | polymorphism |
| Exon 1 | (SEQ ID NO: 1) | (SEQ ID NO: 2) | (SEQ ID NO: 3) |
| Exon 4 | (SEQ ID NO: 4) | (SEQ ID NO: 5) | (SEQ ID NO: 6) |
| Exon 9 | (SEQ ID NO: 7) | (SEQ ID NO: 8) | (SEQ ID NO: 9) |
| Exon 14 | (SEQ ID NO: 10) | (SEQ ID NO: 11) | (SEQ ID NO: 12) |
| Intronic | (SEQ ID NO: 13) | (SEQ ID NO: 14) | N/a |

PCR Conditions:
18 μl ABgene 2 mM Reddy load including Taq polymerase
1 μl 5 μM primer pair mix
1 μl genomic DNA (various individuals)
   PCR Programme:
94° C. 1 min; (94° C. 30 sec, 58° C. 30 sec, 72° C. 2 min) for 34 cycles; 72° C. 10 min.
   2. OATPF Polymorphisms

Sequencing of DNA from 20 individuals identified the following 5 polymorphisms in the OATPF DNA sequence:

| | | | | Protein | |
|---|---|---|---|---|---|
| | | | | sequence | |
| Position of SEQ | | | Resultant codon | SEQ ID NO | Frequency |
| ID NO 16 | Region | Variation | change | 17 | variant allele |
| 86 | Exon 1 | A/G | aat-gat | Asn8Asp | 0.03G |
| 505 | Exon 4 | C/T | N/a | Ser147Ser | 0.46T |
| 1339 | Exon 9 | A/G | N/a | Ala425Ala | 0.05G |
| 1991 | Exon 14 | C/T | N/a | Leu643Leu | 0.25T |

| Position | | | | | |
|---|---|---|---|---|---|
| of SEQ | | | Resultant | Protein | |
| ID NO: | | | codon | sequence | |
| 15 | Region | Variation | change | SEQ ID NO 17 | Frequency variant allele |
| 11-16 | Intronic | TAAAAA/ | N/a | N/a | 0.06ACTTTGAAAG |
| | | ACTTTGA | | | |
| | | AAG | | | |

1. A method for the detection of a polymorphism in OATPF in a human which method comprises determining the sequence of the human at any one of the following positions:
position 11-16 of SEQ ID NO: 15;
positions 86, 505, 1339 and 1991 of SEQ ID NO: 16;
position 8 of SEQ ID NO: 17.

2. A method according to claim 1 wherein the polymorphism is further defined as:
polymorphism at position 11-16 is presence of TAAAAA and/or insertion of ACTTTGAAAG in lieu thereof;
polymorphism at position 86 is presence of A and/or G;
polymorphism at position 505 is presence of C and/or T;
polymorphism at position 1339 is presence of A and/or G;
polymorphism at position 1991 is presence of A and/or T; and
polymorphism at position 8 is presence of Asn and/or Asp.

3. A method according to claim 1 wherein the method for detection of a nucleic acid polymorphism is selected from amplification refractory mutation system and restriction fragment length polymorphism.

4. Use of a method defined in claim 1 to assess the pharmacogenetics of a drug transportable by OATPF.

5. A polynucleotide comprising at least 20 contiguous bases of the human OATPF gene and comprising an allelic variant selected from any of the following:

| Region | variant | Position |
|---|---|---|
| Exon 1 | G | 86 (SEQ ID NO: 16) |
| Exon 4 | T | 505 (SEQ ID NO: 16) |
| Exon 9 | G | 1339 (SEQ ID NO: 16) |
| Exon 14 | T | 1991 (SEQ ID NO: 16) |
| Intronic | ACTTTGAAAG | 11-20 (SEQ ID NO: 15) |

6. An allele specific primer capable of detecting an OATPF gene polymorphism at position 11-16 of SEQ ID NO: 15, and position 86, 505, 1339 and 1-991 of SEQ ID NO: 16.

7. An allele specific oligonucleotide probe capable of detecting a OATPF gene polymorphism at position 11-16 of SEQ ID NO: 15 and position 86, 505, 1339 and 1991 of SEQ ID NO: 16.

8. A diagnostic kit comprising the allele-specific primer of claim 6.

9. A method of treating a human in need of treatment with a drug transportable by OATPF in which the method comprises:
i) detection of a polymorphism in OATPF in a human, which method comprises determining the sequence of the human at one or more of:
position 11-16 of SEQ ID NO: 15;
positions 86, 505, 1339 and 1991 of SEQ ID NO: 16;
position 8 of SEQ ID NO: 17; and
ii) administering an effective amount of the drug.

10. Use of a drug transportable by OATPF in preparation of a medicament for treating a disease in a human determined as having a polymorphism at one of the following positions:
position 11-16 of SEQ ID NO: 15;
positions 86, 505, 1339 and 1991 of SEQ ID NO: 16;
position 8 of SEQ ID NO: 17.

11. An allelic variant of human OATPF polypeptide comprising an aspartic acid at position 8 of SEQ ID NO: 17 or a fragment thereof comprising at least 10 amino acids provided that the fragment comprises the allelic variant at position 8 of SEQ ID NO: 17.

12. An antibody specific for an allelic variant of human OATPF polypeptide as described herein having an aspartic acid at position 8 of SEQ ID NO: 17 or a fragment thereof comprising at least 10 amino acids provided that the fragment comprises the allelic variant at position 8 of SEQ ID NO: 17.

13. A diagnostic kit comprising an antibody of claim 12.

14. A diagnostic kit comprising the allele specific oligonucleotide probe of claim 7.
